(19) United States
(12) Patent Application Publication
(10) Pub. No.: US 2005/0261637 A1
(43) Pub. Date: Nov. 24, 2005

(54) T-port with swabbable valve

(75) Inventor: Pavel T. Miller, North St. Petersburg, FL (US)

Correspondence Address: HOLLAND & KNIGHT LLP, ATTN: STEFAN V. STEIN/ IP DEPT., POST OFFICE BOX 1288, TAMPA, FL, 33601-1288, US

(73) Assignee: Halkey-Roberts Corporation

(21) Appl. No.: 10/956,947

(22) Filed: Oct. 1, 2004

Related U.S. Application Data
(60) Provisional application No. 60/573,671, filed on May 21, 2004.

(51) Int. Cl. A61M 5/14

(52) U.S. Cl. 604/256; 251/149.3

(57) ABSTRACT

A slit-type T-port site that includes a swabbable valve with a slit at an end thereof. The valve stem with a slit is located in a valve body and is deformable. When engaged by a tip of an instrument, the top portion of the stem folds inward and its slit opens to form a direct non-hemolytic flow pathway between the instrument and the main T-port site flow channel.

### CROSS-REFERENCE TO RELATED APPLICATIONS

This application claims the benefit of provisional application No. 60/573,671, filed May 21, 2004, the disclosure of which is incorporated by reference herein.

### BACKGROUND OF THE INVENTION

1. Field of the Invention

This invention relates to valves. More particularly, this invention relates to swabbable valves used in the medical industry.

2. Description of the Background Art

As the term implies, the term “swabbable” is often used in conjunction with medical valves having a female luer fitting configured in such a way that allows swabbing of the exposed portion of the valve just prior to connection with a male luer needleless syringe or other device.

Representative swabbable valves are disclosed in U.S. Pat. Nos. 6,036,171, 6,692,478, 6,221,065, 6,168,137, 6,117,114, 6,651,956, 6,113,068 and RE 37,357, the disclosures of which are hereby incorporated by reference herein.

As taught by the above-referenced patents, swabbable valves are often employed in IV-sets for needleless interconnection of an IV bag and its associated tubing to deliver medicine to a patient intravenously. Such needleless interconnection occurs with each device being connected end to end.

Swabbable valves preferably satisfy main requirements. For example, they should safely withstand, without loss of performance, at least 100 connects and disconnects to an injection site before the set is replaced. In addition, a connection is maintained for an extended period of time before disconnection is made. The site should still be capable of accepting subsequent connections without allowing any leakage. Valves should seal against pressurized fluid within a set. They should withstand pressures in excess of, for example, 30 PSI for a short time, such as during an injection made through an adjacent site or if a pump is connected in the circuit. Further, valves should be capable of being manufactured at high speeds and low costs. At the same time, the design must allow for minimal manufacturing defects. Still further, it is desirable that such valves have as few components as possible, and be easily assembled, without requiring any difficult component orientation or positioning.

With particular regard to medical applications, valves should not contain any dead space where fluid can collect and not be readily flushed away. Also, priming volume should be minimized. Furthermore, valves should also be easily accessible by standard luer connectors and provide secure locking features, so they could be left connected to the site without further assistance from a practitioner.

Another highly-desirable feature is easy and safe swabbability of the valve inlet area. Unfortunately, most current swabbable valves restrict free flow of passing fluid by employing narrow passages, ribs or internal cannula-like features. Restricting the flow path in such a manner may create conditions for hemolytic damage. Such restrictions also make the valve generally more difficult to flush.

Indeed, in valves used for blood sampling, there is a need for valves that do not have any space where fluid could collect and stagnate. The valve should be fully flushable to preclude thrombosis that might otherwise occur in dead spaces even of minimal sizes.

Furthermore, in medical applications, it is usually desirable to prevent the patient from being exposed to the fluid which is being injected to or extracted from the patient, and it is desirable to insulate nurses and doctors from exposure to the liquid which may contain the patient's blood or waste products. However, often the instrument used to inject or withdraw the fluid (which is generally the male component of the syringe), retains some of the fluid on the tip thereof, thus providing a risk to nurses and doctors of being exposed to the fluid. Wiping off this fluid prior to disconnecting the instrument is highly desirable.

As taught for example in U.S. Pat. Nos. 6,221,065 and 6,117,114, the disclosures of which are incorporated by reference herein, Y-site connectors are commonly used in IV-sets. U.S. Pat. RE 37,357 describes a valve in the form of a T-port where resulting flow from the swabbable end is very limited. Such resistance to the flow creates an undesirable condition for leaks around the access instrument's tip. A swabbable valve used as an injection port or a sampling port should pose minimum resistance to the flow from the syringe or communicating line. Restrictive valve geometry means slow fluid delivery and if there is blood in the fluid, there is the possibility of hemolytic damage caused by high flow speeds at narrow or curved passages.

There presently exists a need for a swabbable valve providing in-line access to IV tubing that is particularly suited for use as a sampling and injection site, such as on hemodialysis sets, with minimal obstructions to the blood flow and no dead space or hard-to-flush-out space.

Therefore, it is an object of this invention to provide an improvement which overcomes the aforementioned inadequacies of the prior art devices and provides an improvement which is a significant contribution to the advancement of the swabbable valve art.

Another object of this invention is to provide a swabbable valve allowing needleless in-line access to IV tubing.

Another object of this invention is to provide a swabbable valve incorporated into a T-port facilitating needleless in-line access to medical tubing.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

In preferred aspects, this invention comprises a T-port that has incorporated therein a swabbable valve allowing needleless in-line access to medical tubing or other devices connected thereto. The T-port is particularly suitable for use as a sampling and injection site. Preferably, the T-port is non-hemolytic, presents minimal obstructions to the blood flow, requires minimal priming volume and is non-thrombogenic.

More particularly, the T-port of the invention comprises a T-port body having a longitudinal tubular portion with opposing ends each adapted to sealingly receive the end of medical tubing and a transverse valve portion having a swabbable valve incorporated therein. The T-port preferably incorporates a valve that seals itself to restrict fluid flow therein and decreases the risk of contaminants such as bacteria collecting on or within the valve into the T-port geometry. The T-port is configured such that all external surfaces in the proximity of the valve stem are accessible to be wiped clean with a sterile swab. Further, the T-port includes a stem that provides a relatively flat and wrinkle-free top surface which can be easily swabbed. The T-port minimally restricts fluid flow therethrough without requiring fluid to pass narrow cannula-like passages and also without any rib-like housing features, to thereby provide a valve structure with an unobstructed flow path allowing for smooth fluid flow without hemolytic damage without presenting any difficulty for molding and high speed assembly. The valve structure includes a female valve component that seals with a male component or instrument when the instrument is engaged therewith so that there is no leakage of fluid. The valve structure may include a female valve that obtains an effective seal and does not have a tendency to leak fluid into the surrounding area upon the male component or instrument being disengaged therefrom. Finally, the valve structure allows fluid flow in both directions upon a male component or instrument being engaged therewith.

During use, the T-port may be positioned in-line with a length of medical tubing by fitting the respective ends of the tubing into the opposing ends of the tubular portion.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is an end view of the slit-type T-port site of the invention;

FIG. 2 is a cross-sectional view of FIG. 1, along lines A-A;

FIG. 3 is a cross-sectional view of FIG. 1 along lines A-A where the valve is accessed by a luer;

FIG. 4 is a cross-sectional view of FIG. 1 along lines A-A with tubing attached; and

FIG. 5 is an isometric view of the slit-type T-port site of the invention.

Similar reference characters refer to similar parts throughout the several views of the drawings.

### DETAILED DESCRIPTION OF THE PREFERRED EMBODIMENT

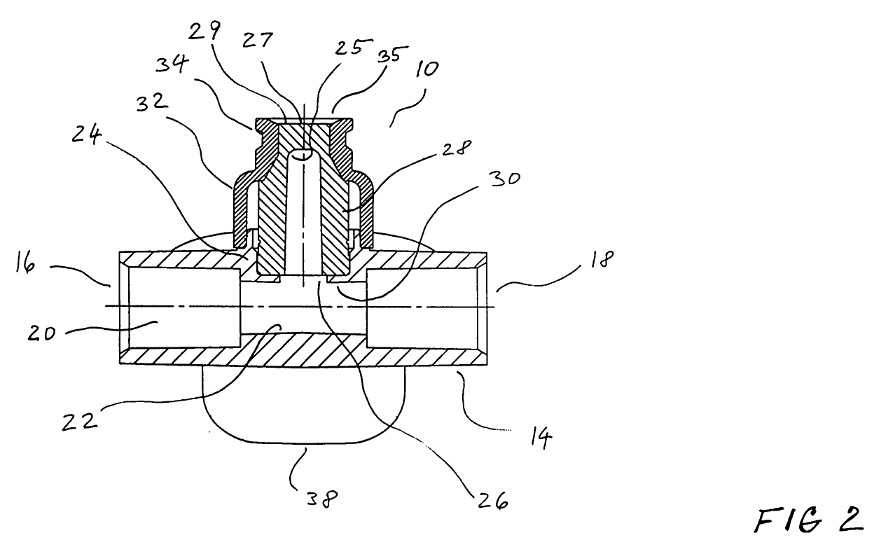

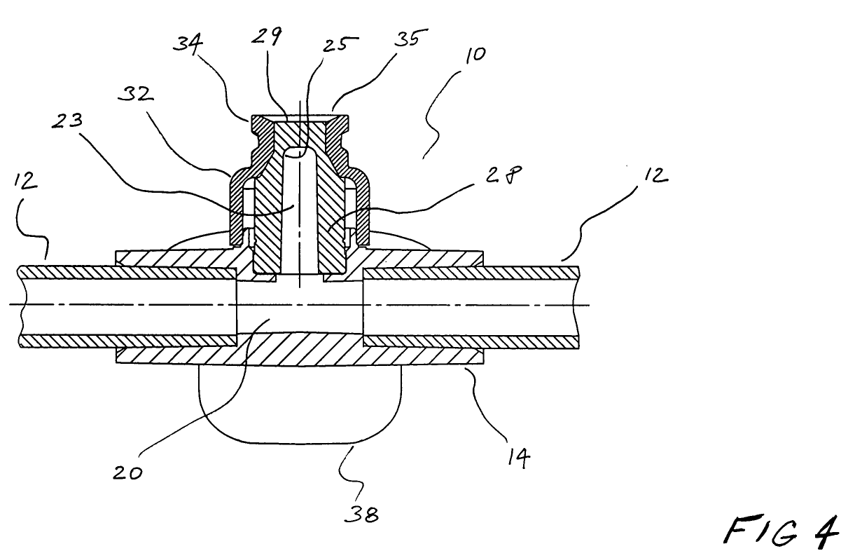

As shown in FIG. 4, the T-port valve 10 of the invention is configured for connection in-line with a length of medical tubing 12. As best shown in FIG. 2, the T-port site 10 of the invention comprises a longitudinal tubular port body 14 having opposing ends 16 and 18. A longitudinal bore 20 extends longitudinally through the port body 14 to fluidly interconnect the ends 16 and 18. The ends 16 and 18 are configured, preferably circular-cylindrically, to sealingly receive the ends of the tubing 12. The longitudinal bore 20 may include a reduced diameter portion 22 in the middle portion of the port body 14 to serve as a stop for the ends of the tubing 12 and to provide an increased wall thickness at such middle portion to which is integrally formed a transverse valve portion 24. A bore 26 extends through the valve portion 24 to be in fluid communication with the bore 20 of the tubular port body 14.

A valve stem 28 is positioned concentrically within the valve portion 24 and retained into position by means of a step 30 formed inwardly from the valve portion 24. A valve cap 32 having a female luer lock fitting feature 34 formed on the upper end thereof is sonically welded or adhesive bonded to the valve portion 24 to entrain the valve stem 24 into sealing position.

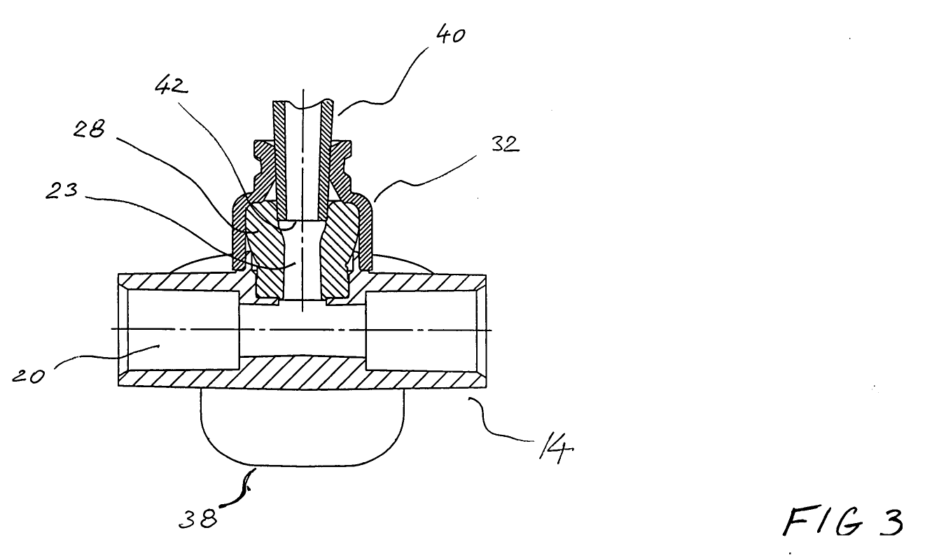

The valve stem 28 includes a generally dome-shaped configuration adapted to sealingly engage against the lumen of the upper portion of the valve cap 32. This sealing engagement and the other functional aspects of the valve stem 28 are more particularly described in the prior patent, U.S. Pat. No. 6,651,956, the disclosure of which is hereby incorporated by reference herein. As disclosed therein and best shown in FIG. 3, upon insertion of the male luer 40 of a needleless medical syringe or other device, the valve stem 28 is compressed inwardly whereupon the dome-shaped end thereof is separated from the lumen of the cap 32. As the tip 42 of the instrument luer 40 is pushed in the stem 28, the slit 27 eventually deforms or opens to allow entry of the tip 42 of the instrument 40, as shown in FIG. 3, and due to the resiliency of stem 28, a tight hermetic seal is formed between the stem 28 and the tip 42 of the instrument luer 40. Valve stem 28 top end 29 collapses and folds inward into the cavity 23, approximately around a fulcrum point 25 located at the area of minimal wall thickness. The slit 27 fully opens and fluid flow is allowed through the stem 28, to or from the instrument 40. The direct non-hemolytic flow pathway is thus formed between the instrument 40 and the main T-port site flow channel 20.

Simultaneously, the needleless medical syringe or other device luer 40 is securely fitted via female 34 to the T-port site 10. Upon disengagement of the luer fitting to remove the medical syringe or other device, the valve stem 28, by virtue of its inherent memory, returns to its sealing engagement with its upper dome portion in sealing engagement with the lumen of the cap 32. Additionally, the stem 28 wipes the tip 42 of the luer 40 clean upon its removal.

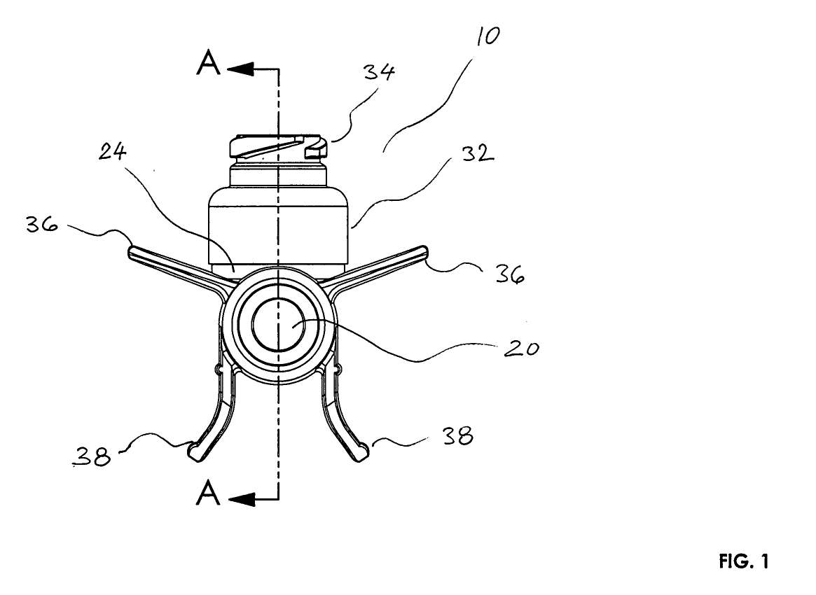

When there is no instrument engaged with the valve (as shown in FIGS. 1, 2, 4 and 5), the slit 27 in the end 29 of the valve stem 28 is fully closed, and top end 29 of the valve stem 28 is generally flush with, or projects axially slightly past, the bottom of the concave area 35 of the valve body 32, allowing the top end 29 of the stem 28 and adjacent areas to be cleaned. This feature is important in medical applications where bacteria growth is to be avoided. To this end, a sterilizing swab can be used to clean the top end 29 of the stem 28 and adjacent areas. Concave area 35 helps to guide an instrument luer 40 into the valve.

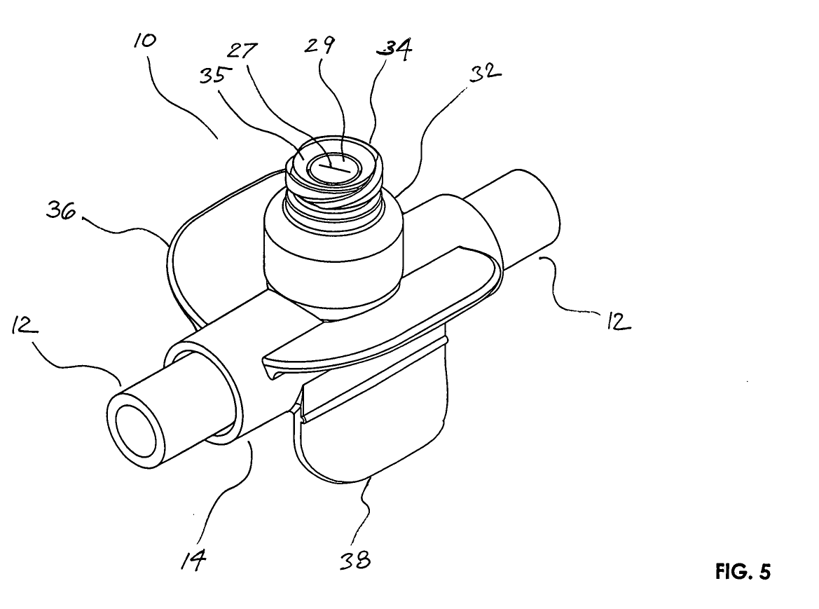

Finally, as best shown in FIGS. 1 and 5, the tubular port body 14 may include a pair of downwardly depending leg flanges 38 and a pair of upwardly extending arm flanges 36 which ergonomically facilitate the holding of the T-port valve by a technician's hand while tactically facilitating the luer-fitting connection of the medical syringe or other device with the technician's other hand.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

1. A T-port valve, comprising in combination:
a longitudinal tubular body having opposing ends and a longitudinal bore extending longitudinally therethrough to fluidly interconnect said ends;
a valve body including a bore extending therethrough in fluid communication with said longitudinal bore of said tubular body;
a valve element positioned concentrically within said valve body;
a valve cap connected to said valve body to entrain said valve element into sealing position; and
said valve element comprising a generally dome-shaped end adapted to sealingly engage against a lumen of an upper portion of said valve cap, said valve element being compressible inwardly to separate said dome-shaped end from said lumen of said cap to thereby open said T-port valve.

2. The T-port valve as set forth in claim 1, wherein said valve body includes a luer fitting allowing a needleless medical syringe or other device to be luer-fitted to said T-port valve to compress said valve element and thereby open said T-port valve and allowing disengagement of the luer fitting to remove the medical syringe or other device whereupon said valve element returns to sealing engagement with said upper dome portion in sealing engagement with said lumen of said cap.

3. The T-port valve as set forth in claim 1, wherein said valve cap is sonically welded to said valve body to entrain said valve element into sealing position.

4. The T-port valve as set forth in claim 1, wherein said valve cap is adhesive bonded to said valve body to entrain said valve element into sealing position.

5. The T-port valve as set forth in claim 1, further including a step formed inwardly from said valve body.

6. The T-port valve as set forth in claim 1, wherein said longitudinal bore includes a reduced diameter portion in said tubular body to serve as a stop for an end of tubing and to provide an increased wall thickness at a middle portion thereof to which is integrally formed said valve body.

7. The T-port valve as set forth in claim 2, wherein said tubular body includes at least one downwardly depending leg flanges and at least one upwardly extending arm flanges which ergonomically facilitates holding of the T-port valve by a technician's hand while tactically facilitating the connection of the medical syringe or other device with the technician's other hand.

8. A slit-type T-port site incorporating a port for in-line connection with medical tubing and a slit-type swabbable valve comprising a swabbable valve body positioned perpendicular to the main port body and a resilient, deformable valve stem which is located in said valve body and wherein the valve body and valve stem are uniformly round and have a uniformly round cross-sectional profile, said valve stem having a linear slit at one end and having an opening at the other end, wherein the valve stem is configured to provide direct connection by the straight cylindrical flow channel to the port main flow channel, wherein the second end of said valve stem is engaged inside said valve body such that said the second end does not shift during actuation of the valve, wherein the slit end is readily accessible for cleaning, wherein said slit is normally closed.

8. A slit-type T-port site as recited in claim 8, where the valve is configured to be actuated via engagement with a male component or instrument, wherein when the valve is actuated, the valve stem shifts in the valve body, and the slit of the valve stem seals against an outer surface of the male component or instrument, thus allowing liquid to flow directly through the valve stem.

9. A slit-type T-port site as recited in claim 8, wherein the valve is configured such that when the valve is actuated, fluid could flow in either direction through the valve.

10. A slit-type T-port site as recited in claim 8, wherein the valve is configured such that when the valve is not actuated, the slit in the valve stem is closed, thereby preventing fluid from leaking into or out of an end of the valve stem.

11. A slit-type T-port site as recited in claim 8, wherein the valve is configured to be actuated via engagement with a male component or instrument, and wherein the valve stem is configured to swab or clean a tip of the male component or instrument upon the tip being removed from the slit.

12. A slit-type T-port site as recited in claim 8, wherein the valve includes a fluid flow area and neither the valve body nor the valve stem includes any ribs which are disposed in the fluid flow area.

13. A slit-type swabbable site as recited in claim 8, wherein the end of the stem is flat and wrinkle-free.

14. A slit-type T-port site as recited in claim 8, wherein said valve body has an end which provides an opening in which sits the valve stem, said end of said valve body providing a concave area which leads into the valves.

15. A slit-type T-port site incorporating a port for in-line connection with medical tubing and a slit-type swababble valve comprising a swabbable valve body positioned perpendicular to main port body and a resilient deformable valve stem which is located in said valve body and wherein the valve body and valve stem are uniformly round and have a uniformly round cross-sectional profile, said valve stem having a linear slit at one end and having an opening at the other end, wherein said valve stem is configured to provide direct connection by the straight cylindrical flow channel to the port main flow channel, wherein the second end of said valve stem is engaged inside said valve body such that said second end does not shift during actuation of the valve, wherein slit end is readily accessible for cleaning, wherein said slit is normally closed, wherein the valve stem is configured to collapse and fold inward generally about a fulcrum point which is located at an area of minimal wall thickness.

16. A slit-type T-port site as recited in claim 15, where the valve is configured to be actuated via engagement with a male component or instrument, wherein when the valve is actuated, the valve stem shifts in the valve body, and the slit of the valve stem seals against an outer surface of the male component or instrument, thus allowing liquid to flow directly through the valve stem.

17. A slit-type T-port site as recited in claim 15, wherein the valve is configured such that when the valve is actuated, fluid could flow in either direction through the valve.

18. A slit-type T-port site as recited in claim 15, wherein the valve is configured such that when the valve is not actuated, the slit in the valve stem is closed, thereby preventing fluid from leaking into or out of an end of the valve stem.

19. A slit-type T-port site as recited in claim 15, wherein the valve is configured to be actuated via engagement with a male component or instrument, and wherein the valve stem is configured to swab or cleans a tip of the male component or instrument upon the tip being removed from the slit.

20. A slit-type T-port site as recited in claim 15, wherein the valve includes a fluid flow area and neither the valve body nor the valve stem includes any ribs which are disposed in the fluid flow area.

21. A slit-type swabbable site as recited in claim 15, wherein the end of the stem is flat and wrinkle-free.

22. A slit-type T-port site as recited in claim 15, wherein said valve body has an end which provides an opening in which sits the valve stem, said end of said valve body providing a concave area which leads into the valve.
